(19) United States
(12) Patent Application Publication
(10) Pub. No.: US 2010/0268315 A1
(43) Pub. Date: Oct. 21, 2010

(54) Castellated Sleeve Stent-Graft Delivery System and Method

(75) Inventors: Brian Glynn, Santa Rosa, CA (US); Mark L. Stiger, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/426,020

(22) Filed: Apr. 17, 2009

(51) Int. Cl. A61F 2/06

(52) U.S. Cl. 623/1.11

(57) ABSTRACT

A method of deploying a stent graft includes radially constraining proximal apexes of a proximal anchor stent ring of the stent-graft in a space between merlons of a castellated sleeve of a tip and a spindle having spindle pins, the proximal apexes extending around the spindle pins. A graft material of the stent-graft is radially constrained in a primary sheath, a proximal end of the graft material being attached to distal apexes of the proximal anchor stent ring. The proximal anchor stent ring further includes struts extending between the proximal apexes and the distal apexes. The primary sheath is retracted to allow the proximal end of the graft material and the distal apexes of the proximal anchor stent ring to pivot out beyond the confines of the sleeve to a large angle as the proximal end radially expands. The struts then extend through embrasures of the castellated sleeve. The tip is advanced to deploy the proximal apexes.

### BACKGROUND OF THE INVENTION

1. Field of the Invention

This invention relates generally to medical devices and procedures, and more particularly to a method and system of deploying a stent-graft in a vascular system.

2. Description of the Related Art

Prostheses for implantation in blood vessels or other similar organs of the living body are, in general, well known in the medical art. For example, prosthetic vascular grafts formed of biocompatible materials (e.g., Dacron or expanded, porous polytetrafluoroethylene (PTFE) tubing) have been employed to replace or bypass damaged or occluded natural blood vessels.

A graft material supported by a framework is known as a stent-graft or endoluminal graft. In general, the use of stent-grafts for treatment or isolation of vascular aneurysms and vessel walls which have been thinned or thickened by disease (endoluminal repair or exclusion) is well known.

Many stent-grafts, are “self-expanding”, i.e., inserted into the vascular system in a compressed or contracted state, and permitted to expand upon removal of a restraint. Self-expanding stent-grafts typically employ a wire or tube configured (e.g., bent or cut) to provide an outward radial force and employ a suitable elastic material such as stainless steel or nitinol (nickel-titanium). Nitinol may additionally employ shape memory properties.

The self-expanding stent-graft is typically configured in a tubular shape of a slightly greater diameter than the diameter of the blood vessel in which the stent-graft is intended to be used. In general, rather than inserting in a traumatic and invasive manner, stents and stent-grafts are typically deployed through a less invasive intraluminal delivery, i.e., cutting through the skin to access a lumen or vasculature or percutaneously via successive dilatation, at a convenient (and less traumatic) entry point, and routing the stent-graft through the lumen to the site where the prosthesis is to be deployed.

Intraluminal deployment in one example is effected using a delivery catheter with coaxial inner tube, sometimes called an inner tube (plunger), and an outer tube, sometimes called the sheath, arranged for relative axial movement. The stent-graft is compressed and disposed within the distal end of the sheath in front of the inner tube.

The catheter is then maneuvered, typically routed though a vessel (e.g., lumen), until the end of the catheter containing the stent-graft is positioned in the vicinity of the intended treatment site. The inner tube is then held stationary while the sheath of the delivery catheter is withdrawn. The inner tube prevents the stent-graft from moving back as the sheath is withdrawn.

As the sheath is withdrawn, the stent-graft is gradually exposed from a proximal end to a distal end of the stent-graft, the exposed portion of the stent-graft radially expands so that at least a portion of the expanded portion is in substantially conforming surface contact with a portion of the interior of the blood vessel wall.

The proximal end of the stent-graft is the end closest to the heart by way of blood flow path whereas the distal end is the end furthest away from the heart during deployment. In contrast and of note, the distal end of the catheter is usually identified to the end that is farthest from the operator (handle) while the proximal end of the catheter is the end nearest the operator (handle). For purposes of clarity of discussion, as used herein, the distal end of the catheter is the end that is farthest from the operator (the end furthest from the handle) while the distal end of the stent-graft is the end nearest the operator (the end nearest the handle or the handle itself), i.e., the distal end of the catheter and the proximal end of the stent-graft are the ends furthest from the handle while the proximal end of the catheter and the distal end of the stent-graft are the ends nearest the handle. However, those of skill in the art will understand that depending upon the access location, the distal and proximal end descriptors for the stent-graft and delivery system description may be consistent or opposite in actual usage.

Many self-expanding stent-graft deployment systems are configured to have each exposed increment of the stent graft at the proximal end of the stent-graft deploy (flare out or mushroom) as the sheath is pulled back. The proximal end of the stent-graft is typically designed to expand to fixate and seal the stent-graft to the wall of the vessel during deployment. Such a configuration leaves little room for error in placement since re-positioning the stent-graft after initial deployment, except for a minimal pull down retraction, is usually difficult if possible at all. The need to achieve accurate proximal end positioning of the stent-graft first makes accurate pre-deployment positioning of the stent-graft critical.

Attempts to overcome this problem generally fail to provide adequate control in manipulating the stent-graft positioning in both the initial deployment of the stent-graft and the re-deployment of the stent-graft (once the stent-graft has been partially deployed).

Another problem encountered with existing systems, particularly with systems that have a distal end of a stent-graft fixed during deployment (or during the uncovering of the sheath) is the frictional forces that can cause the stent-graft to axially compress or bunch up as the sheath is retracted. This bunching increases the density of the stent-graft within the sheath and can further increase the frictional drag experienced during deployment.

### SUMMARY OF THE INVENTION

A method of deploying a stent-graft includes radially constraining proximal apexes of a proximal anchor stent ring of the stent-graft in a space between merlons of a castellated edge of a sleeve of a tip and a spindle having spindle pins, the proximal apexes extending around the spindle pins. A graft material of the stent-graft is radially constrained in a primary sheath, a proximal end of the graft material being attached to distal apexes of the proximal anchor stent ring. The proximal anchor stent ring further includes struts extending between the proximal apexes and the distal apexes.

The primary sheath is retracted to allow the proximal end of the graft material and the distal apexes of the proximal anchor stent ring to radially expand, wherein the struts extend through embrasures of the castellated edge of the sleeve.

Proximal (or bare) spring struts are unrestrained and released through the embrasures. This allows the proximal end of the graft material and the distal apexes of the proximal spring to radially expand and engage a vessel wall of a vessel in which the stent-graft is being deployed.

By engaging the proximal end of the graft material with the vessel wall, accurate positioning of the stent-graft is achieved. For example, the stent-graft is used in a thoracic aortic application where it is important that the proximal end of the graft material engage the vessel wall prior to complete deployment of the stent-graft. The tip is then advanced to deploy the proximal apexes (apices) of the stent-graft.

These and other features according to the present invention will be more readily apparent from the detailed description set forth below taken in conjunction with the accompanying drawings.

### BRIEF DESCRIPTION OF THE DRAWINGS

FIG. 1 is a schematicized partial cross-sectional view of a stent-graft delivery system without a stent-graft and outer sheath in accordance with one embodiment;

FIG. 2 is a schematicized perspective view of a tapered tip of the stent-graft delivery system of FIG. 1;

FIG. 3 is an enlarged perspective view of the region III of a castellated sleeve of the tapered tip of FIG. 2;

FIG. 4 is a schematicized partial cross-sectional view of the stent-graft delivery system of FIG. 1 including a stent-graft located within a retractable primary sheath in a pre-deployment un-retracted position;

FIG. 5 is a schematicized partial cross-sectional view of the stent-graft delivery system of FIG. 4 with the retractable primary sheath partially retracted;

FIG. 6 is an enlarged perspective view of the region VI of the stent-graft delivery system of FIG. 5;

FIG. 7 is a partial end view viewed from the perspective of arrow VII of FIG. 6 of a merlon and a pair of anchor pins;

FIG. 8 is a schematicized partial cross-sectional view of the stent-graft delivery system of FIG. 5 after deployment of a proximal anchor stent ring of the stent-graft;

FIG. 9 is an enlarged perspective view of a region of a stent-graft delivery system similar to the region of the stent-graft delivery system of FIG. 6 in accordance with one embodiment;

FIG. 10 is a partial end view viewed from the perspective of arrow X of FIG. 9 of a merlon and a pair of anchor pins; and

FIG. 11 is an enlarged perspective view of a region of a stent-graft delivery system similar to the region of the stent-graft delivery system of FIG. 6 in accordance with one embodiment.

In the following description, the same or similar elements are labeled with the same or similar reference numbers.

### DETAILED DESCRIPTION

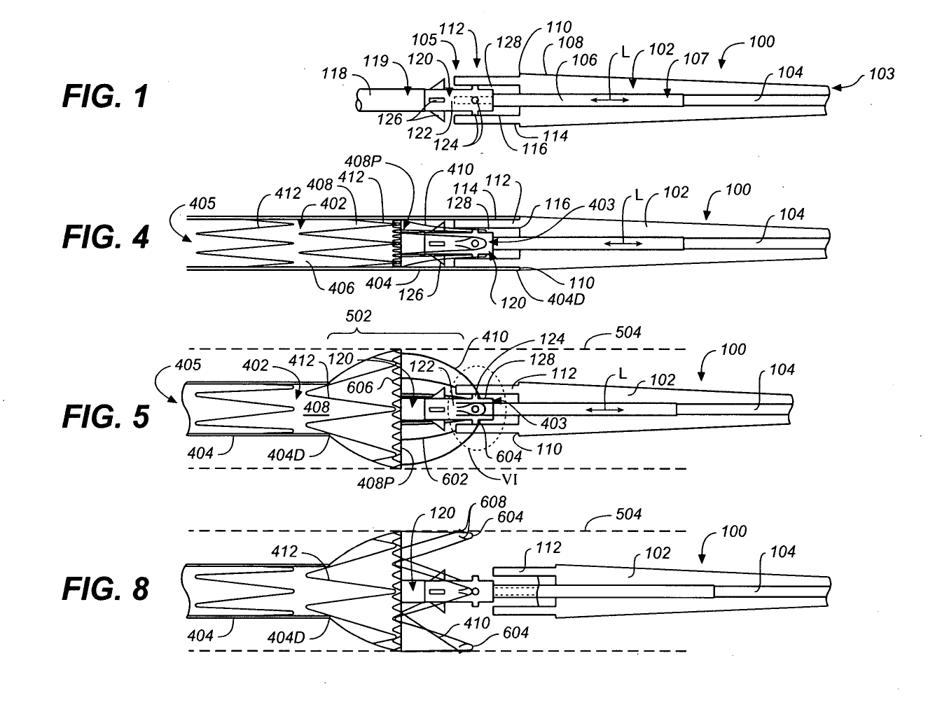

FIG. 1 is a schematicized partial cross-sectional view of a tip of a stent-graft delivery system 100 without a stent-graft and outer sheath in accordance with one embodiment. Stent-graft delivery system 100 includes a tapered tip 102 that is flexible and able to provide trackability in tight and tortuous vessels. Tapered tip 102 includes a guidewire lumen 104 therein for connecting to adjacent members and allowing passage of a guidewire through tapered tip 102. Other tip shapes such as bullet-shaped tips could also be used.

An inner tube 106 also defines a lumen, e.g., a guide wire lumen, therein. A distal end 107 of inner tube 106 is located within and secured to tapered tip 102, i.e., tapered tip 102 is mounted on inner tube 106. As shown in FIG. 1, the lumen of inner tube 106 is in fluid communication with guidewire lumen 104 of tapered tip 102 such that a guide wire can be passed through inner tube 106 and out distal end 107, through guidewire lumen 104 of tapered tip 102, and out a distal end 103 of tapered tip 102.

Tapered tip 102 includes a tapered outer surface 108 that gradually increases in diameter. More particularly, tapered outer surface 108 has a minimum diameter at distal end 103 and gradually increases in diameter proximally, i.e., in the direction of the operator (or handle of stent-graft delivery system 100), from distal end 103.

Tapered outer surface 108 extends proximally to a primary sheath abutment surface (shoulder) 110 of tapered tip 102. Primary sheath abutment surface 110 is an annular ring (shoulder) perpendicular to a longitudinal axis L of stent-graft delivery system 100.

Tapered tip 102 further includes a castellated sleeve 112, sometimes called a castellated tip, extending proximally from primary sheath abutment surface 110. Generally, castellated sleeve 112 is at a proximal end 105 of tapered tip 102. Castellated sleeve 112 extends proximally along the longitudinal axis of the delivery system from primary sheath abutment surface 110. Castellated sleeve 112 includes an outer castellated cylindrical surface 114 and an inner castellated cylindrical surface 116 as discussed further below.

Stent-graft delivery system 100 further includes an outer tube 118 having a spindle 120 located at and fixed to a distal end 119 of outer tube 118. Spindle 120 includes a spindle body 122 having a cylindrical outer surface, a plurality of spindle pins 124 protruding radially outward from spindle body 122, and a plurality of primary sheath guides 126 protruding radially outward from spindle body 122. Primary sheath guides 126 guide the primary sheath into position over castellated sleeve 112 (see FIG. 4 for example).

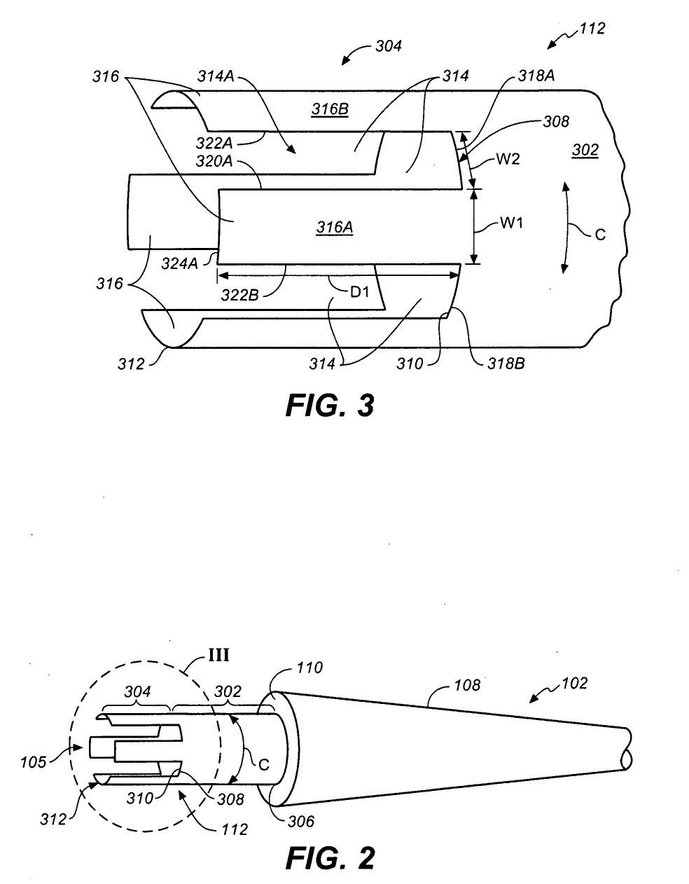

FIG. 2 is a schematicized perspective view of tapered tip 102 of stent-graft delivery system 100 of FIG. 1 in accordance with one example. FIG. 3 is an enlarge perspective view of the region III of castellated sleeve 112 of tapered tip 102 of FIG. 2 in accordance with one example.

Referring now to FIGS. 1, 2, and 3 together, castellated sleeve 112 includes a cylindrical wall portion 302 and a castellation portion 304. Cylindrical wall portion 302 is a hollow cylinder that extends proximally from primary sheath abutment surface 110. More particularly, a distal end 306 of cylindrical wall portion 302 is connected to primary sheath abutment surface 110. Cylindrical wall portion 302 extends proximally from distal end 306 to a proximal end 308 of cylindrical wall portion 302.

Castellation portion 304 is connected to and extends proximally from proximal end 308 of cylindrical wall portion 302. More particularly, a distal end 310 of castellation portion 304 is connected to proximal end 308 of cylindrical wall portion 302. Castellation portion 304 extends proximally from distal end 310 to a proximal end 312 of castellation portion 304.

In one example, cylindrical wall portion 302 and castellation portion 304 are integral, i.e., are a single piece and not a plurality of separate pieces connected together. For example, castellated sleeve 112 is formed by cutting a hypotube.

Castellation portion 304 includes a castellation pattern of embrasures 314 and merlons 316 similar to the distinctive pattern that frames the tops of the walls of many medieval castles, often called battlements. Merlons 316, sometimes called fingers or protrusions, are separated from one another by embrasures 314, sometimes called crenelles or crenels. Similarly, embrasures 314 are separated from one another by merlons 316. Embrasures 314 are openings, sometimes called spaces, within castellated sleeve 112 between merlons 316.

Each embrasure 314 separates adjacent merlons 316 along the circumference C of castellated sleeve 112. To illustrate, a first embrasure 314A of the plurality of embrasures 314 separates a first merlon 316A of the plurality of merlons 316 from a second merlon 316B of the plurality of merlons 316 along circumference C.

Embrasure 314A is defined by a circumferential edge 318A of castellated sleeve 112, a first longitudinal edge 320A of castellated sleeve 112, and a second longitudinal edge 322A of castellated sleeve 112. Circumferential edge 318A extends along the circumference C of castellated sleeve 112 between longitudinal edges 320A, 322A. Circumferential edge 318A is at proximal end 308 of cylindrical wall portion 302 and distal end 310 of castellation portion 304. Longitudinal edges 320A, 322A extend between proximal end 312 of castellation portion 304 and circumferential edge 318A.

To further illustrate, merlon 316A is defined by a circumferential edge 324A of castellated sleeve 112, first longitudinal edge 320A of castellated sleeve 112, and a third longitudinal edge 322B (similar to longitudinal edge 322A) of castellated sleeve 112. Circumferential edge 324A extends along the circumference C of castellated sleeve 112 between longitudinal edges 320A, 322B. Circumferential edge 324A is at proximal end 312 of castellation portion 304 and generally at proximal end 105 of tapered tip 102. Longitudinal edge 320A extends between circumferential edge 324A and circumferential edge 318A at distal end 310 of castellation portion 304. Longitudinal edge 322B extends between circumferential edge 324A and a second circumferential edge 318B similar to circumferential edge 318A at distal end 310 of castellation portion 304.

In accordance with one example, the diameter of outer castellated cylindrical surface 114 is approximately 0.235 inches (5.97 mm) and thus circumference C is approximately 0.738 inches (18.8 mm). Each merlon 316 has a width W1 along circumference C of approximately 0.039 inches (1.00 mm). Accordingly, each embrasure 314 has a width W2 along circumference C of approximately 0.146 inches (3.71 mm). Further, the longitudinal depth D1 of each embrasure 314 and merlon 316 is in the approximate range of 0.118 inches (3.00 mm) to 0.394 inches (10.0 mm).

Although a single embrasure 314A and a single merlon 316A are described in detail above, this description applies generally to the castellation pattern of embrasures 314 and merlons 316. Further, although four merlons 316 are illustrated, in other examples, a tapered tip similar to tapered tip 102 is formed with more or less than four merlons similar to merlons 316, e.g., five to eight merlons.

Generally, the width W2 along the circumference of each embrasure is set forth by the following relation 1:

W2=(C−X(W1))/NE Relation 1

where C is the circumference, X is the number of merlons, W1 is the width of each merlon along the circumference, and NE is the number of embrasures.

As illustrated in FIG. 1, spindle 120 is configured to slip inside of castellated sleeve 112 such that spindle pins 124 are directly adjacent to, or contact, inner castellated cylindrical surface 116 of castellated sleeve 112. Spindle pins 124 extend from spindle body 122 towards and to castellated sleeve 112. More particularly, each spindle pin 124 extends to a respective merlon 316.

Generally, the diameter to which spindle pins 124 extend from spindle body 122 is approximately equal to, or slightly less than, the diameter of inner castellated cylindrical surface 116 of castellated sleeve 112 allowing spindle pins 124 to snugly fit inside of castellated sleeve 112. Space 128 exists between merlons 316 and spindle body 122.

Inner tube 106 is within and extends through outer tube 118 and spindle 120. Inner tube 106 and thus tapered tip 102 is moved along longitudinal axis L (longitudinally moved) relative to outer tube 118 and thus spindle 120 to release the proximal end of a stent-graft as discussed further below. The term “stent-graft” used herein should be understood to include stent-grafts and other forms of endoprosthesis.

FIG. 4 is a schematicized partial cross-sectional view of stent-graft delivery system 100 of FIG. 1 including a stent-graft 402 located within a retractable primary sheath 404 in a pre-deployment un-retracted position.

Primary sheath 404 is a hollow tube and defines a lumen 406 therein through which outer tube 118 and inner tube 106 extend. Primary sheath 404 is in a pre-deployment un-retracted position in FIG. 4. Primary sheath 404 is moved proximally along longitudinal axis L, sometimes called retracted, relative to outer tube 118/spindle 120 and thus stent-graft 402 to deploy a portion of stent-graft 402 as discussed further below.

In one example, stent-graft 402 is a self-expanding stent-graft such that stent-graft 402 self-expands upon being released from its radially constrained position. In accordance with this example, stent-graft 402 includes a graft material 408, e.g., formed of polyester or Dacron material, and a plurality of resilient self-expanding support structures, e.g., formed of super elastic self-expanding memory material such as nitinol. Graft material 408 includes a proximal end 408P.

The support structures include a proximal anchor (bare) stent ring 410 at a proximal end 403 of stent-graft 402 and one or more stent rings 412 distal to proximal anchor stent ring 410. Proximal anchor stent ring 410 is attached at proximal end 408P of graft material 408. Proximal anchor stent ring 410 and stent rings 412 are attached to graft material 408, e.g., by sutures, adhesive, or other means.

As shown in FIG. 4, stent-graft 402 is in a radially constrained configuration over outer tube 118 and spindle 120. Stent-graft 402 is located within and radially compressed by primary sheath 404. Further, proximal apexes, sometimes called crowns, of proximal anchor stent ring 410 of stent-graft 402 are radially constrained and held in position (captured) in spaces 128 between spindle body 122 and merlons 316 of castellated sleeve 112.

Generally, graft material 408 of stent-graft 402 is radially constrained by primary sheath 404 and the proximal apexes of proximal anchor stent ring 410 are radially constrained by castellated sleeve 112 allowing sequential and independent deployment of graft material 408 and proximal apexes of proximal anchor stent ring 410 of stent-graft 402.

Primary sheath 404 includes a distal end 404D adjacent to or in abutting contact with primary sheath abutment surface 110 of tapered tip 102. Distal end 404D fits snugly around castellated sleeve 112 and in one example lightly presses radially inward on outer castellated cylindrical surface 114 of castellated sleeve 112.

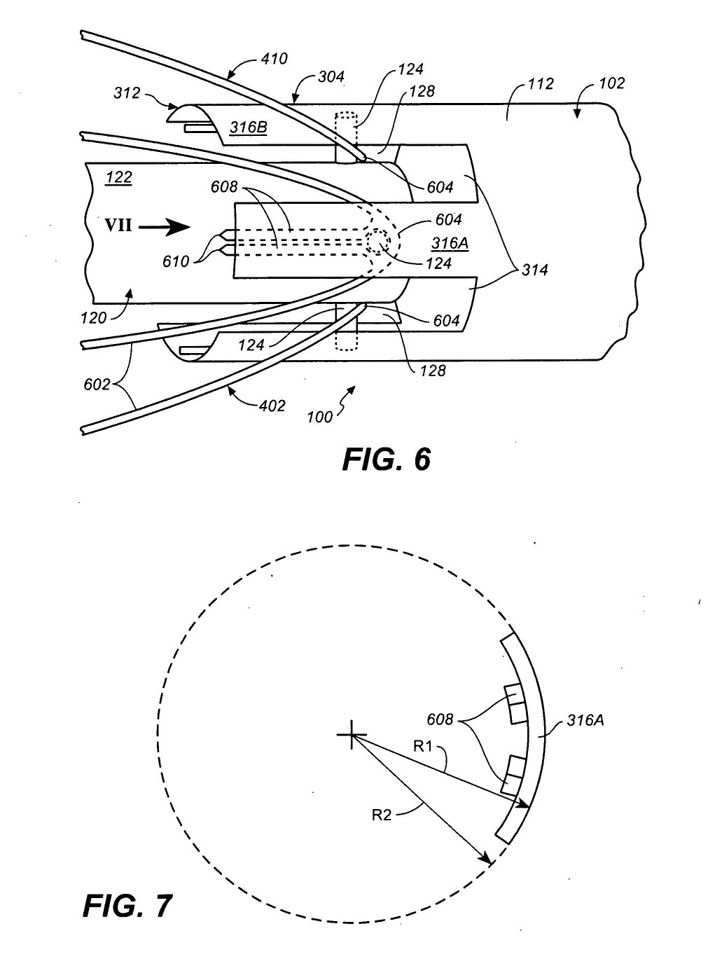

FIG. 5 is a partial cross-sectional view of stent-graft delivery system 100 of FIG. 4 with retractable primary sheath 404 partially retracted. FIG. 6 is an enlarged perspective view of the region VI of stent-graft delivery system 100 of FIG. 5 in accordance with one example. FIG. 7 is a partial end view from the perspective of arrow VII of FIG. 6 of merlon 316A and a pair of anchor pins 608 in accordance with one example.

Referring now to FIGS. 5, 6 and 7 together, primary sheath 404 is partially retracted such that distal end 404D is spaced apart from tapered tip 102. Further, due to the retraction of primary sheath 404, a proximal portion 502 of stent-graft 402 is exposed and partially deployed (expanded). Proximal portion 502 is a portion of stent-graft 402 distal to proximal apexes 604 of proximal anchor stent ring 410 but proximal to the remaining portion of stent-graft 402.

Proximal anchor stent ring 410 includes a zigzag pattern of struts 602 alternating between proximal apexes (crowns) 604 and distal apexes 606 of proximal anchor stent ring 410. Distal apexes 606 are attached to graft material 408 of stent-graft 402.

Proximal anchor stent ring 410 further includes anchor pins (hooks) 608. More particularly, a pair of anchor pins 608 is integral with and located on struts 602 adjacent each proximal apex 604. In accordance with this example, anchor pins 608 include distal tips 610, e.g., sharp points, which facilitate penetration of anchor pins 608 into the wall of the vessel in which stent-graft 402 is deployed as discussed further below with reference to FIG. 8.

As illustrated, proximal apexes 604 of proximal anchor stent ring 410 are radially constrained by merlons 316. More particularly, each proximal apex 604 extends around a spindle pin 124 and is located and secured within space 128 between spindle body 122 and a respective merlon 316.

Further, anchor pins 608 are located and radially constrained within space 128 between spindle body 122 and a respective merlon 316. More particularly, a pair of anchor pins 608 is radially constrained with each respective merlon 316.

As illustrated in FIG. 7, in accordance with this example, the radius R1 of curvature of merlons 316 equals the radius R2 of curvature of castellated sleeve 112. This curvature of merlons 316 facilitates retention of anchor pins 608 under merlons 316. To further facilitate retention of anchor pins 608, in accordance with one example, anchor pins 608 are heat set to remain under merlons 316 during the stage of partial deployment of stent-graft 402 as illustrated in FIGS. 5, 6 and 7.

In contrast, struts 602 are unrestrained and when released pivot out to large angle beyond the confines of the sleeve portion through embrasures 314. More particularly, a pair of struts 602 is aligned with and extends through each embrasure 314. In this manner, proximal anchor stent ring 410, i.e., the distal end thereof, is allowed to radially expand to a diameter larger than the diameter of castellated sleeve 112.

By allowing proximal anchor stent ring 410 to radially expand while only constraining proximal apexes 604, radial expansion of proximal end 408P of graft material 408 and distal apexes 606 to contact and engage a vessel wall 504 is facilitated. By engaging proximal end 408P of graft material 408 with vessel wall 504, accurate positioning of stent-graft 402 is achieve. For example, stent-graft delivery system 100 is used in a thoracic application where it is important that proximal end 408P of graft material 408 engage vessel wall 504 prior to complete deployment of stent-graft 402.

As proximal portion 502 is only partially deployed and proximal apexes 604 of proximal anchor stent ring 410 are radially constrained and un-deployed, stent-graft's initial pre final (proposed) position can be observed and if needed the stent-graft 402 can be repositioned in the event that the initial positioning of stent-graft 402 is less than desirable. More particularly, to reposition stent-graft 402, the retraction of primary sheath 404 is halted. Stent-graft delivery system 100 is then moved to reposition stent-graft 402, for example, stent-graft 402 is rotated or moved proximally or distally without a substantial risk of damaging vessel wall 504 in which stent-graft 402 is being deployed.

Further, as proximal end 403 of stent-graft 402 is secured thus fixing proximal end 403 of stent-graft 402 and keeping it in tension as primary sheath 404 is retracted and, in one example, distal end 405 is free to move within primary sheath 404, bunching of stent-graft 402 during retraction of primary sheath 404 is avoided. By avoiding bunching, frictional drag of stent-graft 402 on primary sheath 404 during retraction is minimized thus facilitating smooth and easy retraction of primary sheath 404.

Once the proximal end of stent-graft 402 is properly positioned, proximal apexes 604 of proximal anchor stent ring 410 are released and deployed securing stent-graft 402 in position within vessel wall 504 as discussed in greater detail below with reference to FIG. 8.

FIG. 8 is a partial cross-sectional view of stent-graft delivery system 100 of FIG. 5 after deployment of proximal anchor stent ring 410 of stent-graft 402. Referring now to FIG. 8, tapered tip 102 is advanced relative to spindle 120 to expose and release proximal apexes 604 of proximal anchor stent ring 410. Upon being released from merlons 316 of castellated sleeve 112 of tapered tip 102, proximal apexes 604 (and generally proximal anchor stent ring 410) self-expand into vessel wall 504 in which stent-graft 402 is being deployed.

Anchor pins 608 penetrate into vessel wall 504 thus anchoring proximal anchor stent ring 410 to vessel wall 504. Accordingly, after deployment and anchoring of proximal anchor stent ring 410 to vessel wall 504, primary sheath 404 is fully retracted to fully deploy stent-graft 402 without migration.

However, in another example, primary sheath 404 is fully retracted prior to release of proximal apexes 604 of proximal anchor stent ring 410. To illustrate, instead of being partially retracted at the stage of deployment illustrated in FIG. 5, primary sheath 404 is fully retracted while proximal apexes 604 of proximal anchor stent ring 410 are still radially constrained.

Further, stent-graft 402 is set forth above as being a self-expanding stent. In accordance with another example, instead of being a self-expanding stent-graft, stent-graft delivery system 100 includes an expansion member, e.g., a balloon, which is expanded to expand and deploy the stent-graft.

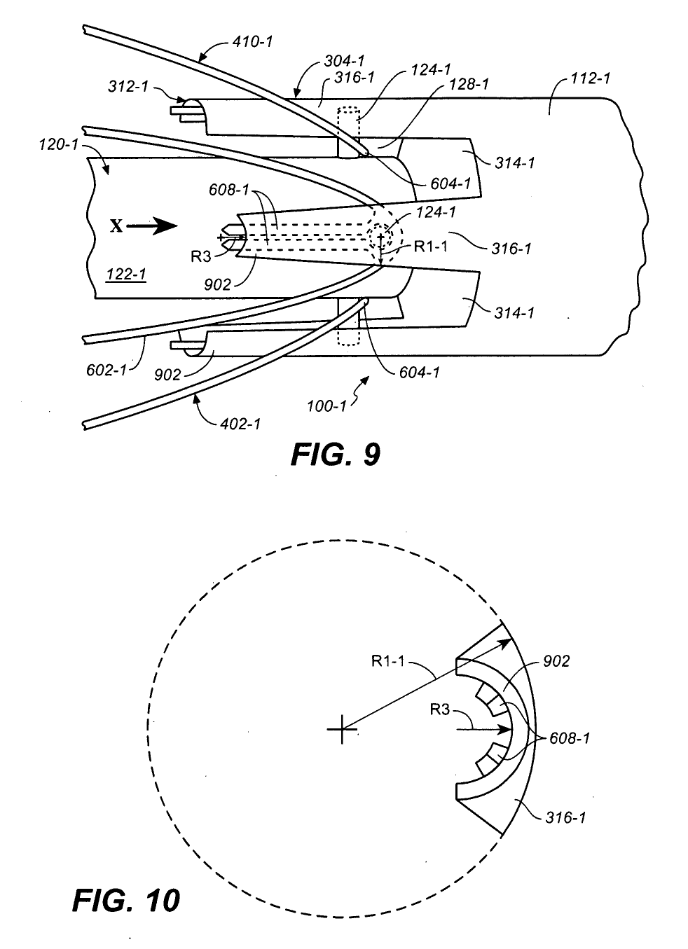

As set forth above in reference to FIG. 7, in one example, the radius R1 of curvature of merlons 316 equals the radius R2 of curvature of castellated sleeve 112. In accordance with one example, a rotation locking feature such as spines (not shown) are provided to prevent rotation of castellated sleeve 112 relative to spindle 120. In accordance with another example as discussed below in reference to FIGS. 9 and 10, the curvature of the merlons is greater than the curvature of the castellated sleeve to enhance retention of the anchor pins and prevent inadvertent rotation of the castellated sleeve.

FIG. 9 is an enlarged perspective view of a region of a stent-graft delivery system 100-1 similar to the region of stent-graft delivery system 100 of FIG. 6 in accordance with one example. FIG. 10 is a partial end view viewed from the perspective of arrow X of FIG. 9 of a merlon 316-1 and a pair of anchor pins 608-1 in accordance with one example.

Castellated sleeve 112-1, spindle 120-1, spindle body 122-1, spindle pins 124-1, space 128-1, castellation portion 304-1, proximal end 312-1, embrasures 314-1, merlons 316-1, stent-graft 402-1, proximal anchor stent ring 410-1, struts 602-1, proximal apexes 604-1, and anchor pins 608-1 of stent-graft delivery system 100-1 of FIGS. 9 and 10 are similar to castellated sleeve 112, spindle 120, spindle body 122, spindle pins 124, space 128, castellation portion 304, proximal end 312, embrasures 314, merlons 316, stent-graft 402, proximal anchor stent ring 410, struts 602, proximal apexes 604, and anchor pins 608 of stent-graft delivery system 100 of FIGS. 6 and 7 and only the significant differences between stent-graft delivery system 100-1 and stent-graft delivery system 100 are set forth below.

Referring now to FIGS. 9 and 10 together, in accordance with this example, merlons 316-1 have a variable radius of curvature along their length. More particularly, merlons 316-1 have a first radius R3 of curvature at distal end 312-1 of castellation portion 304-1 less than a second radius R1-1 of curvature at spindle pins 124-1 and distally to spindle pins 124-1. Further, the radius of curvature of merlons 316-1 gradually increases between first radius R3 of curvature at proximal end 312-1 of castellation portion 304-1, i.e., the minimum radius of curvature, and second radius R1-1 of curvature at spindle pins 124-1 (the maximum radius of curvature). Stated another way, proximal ends 902 of merlons 316-1 are tightly curved (have maximum curvature (minimum radius of curvature)) and gradually flare out, i.e., gradually increase in radius of curvature (gradually reduce in curvature), distally from proximal ends 902 to the curve of the remainder of castellated sleeve 112 at spindle pins 124-1.

By forming merlons 316-1 with tightly curved proximal ends 902, proximal ends 902 wrap around anchor pins 608-1 thus facilitating retention of anchor pins 608-1 within merlons 316-1, which in turn prevents the sleeve 112-1 from rotating with respect to the spindle 120-1.

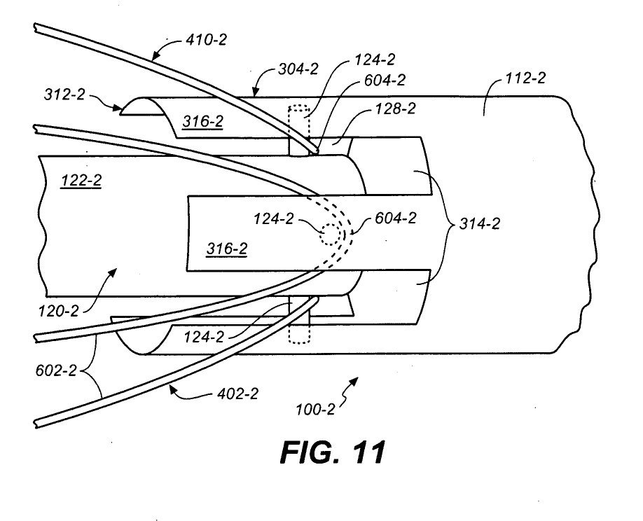

FIG. 11 is an enlarged perspective view of a region of a stent-graft delivery system 100-2 similar to the region of stent-graft delivery system 100 of FIG. 6 in accordance with one example. Castellated sleeve 112-2, spindle 120-2, spindle body 122-2, spindle pins 124-2, space 128-2, castellation portion 304-2, proximal end 312-2, embrasures 314-2, merlons 316-2, stent-graft 402-2, proximal anchor stent ring 410-2, struts 602-2, and proximal apexes 604-2 of stent-graft delivery system 100-2 of FIG. 11 are similar to castellated sleeve 112, spindle 120, spindle body 122, spindle pins 124, space 128, castellation portion 304, proximal end 312, embrasures 314, merlons 316, stent-graft 402, proximal anchor stent ring 410, struts 602, and proximal apexes 604 of stent-graft delivery system 100 of FIG. 6 and only the significant differences between stent-graft delivery system 100-2 and stent-graft delivery system 100 are set forth below.

In accordance with this example, proximal anchor stent ring 410-2 is formed without anchor pins, i.e., without anchor pins similar to anchor pins 608 as illustrated in FIG. 6.

This application is related to Mitchell et al., U.S. Patent Publication 2008-0114442, entitled “DELIVERY SYSTEM FOR STENT-GRAFT WITH ANCHORING PINS”, filed on Nov. 14, 2006, and Mitchell et al., U.S. Patent Application Publication 2008-0114443, entitled “STENT-GRAFT WITH ANCHORING PINS”, filed on Nov. 14, 2006, which are herein incorporated by reference in their entireties.

The drawings and the forgoing description gave examples of embodiments according to the present invention. Numerous variations, whether explicitly given in the specification or not, such as differences in structure, dimension, and use of material, are possible.

What is claimed is:

1. A stent-graft delivery system comprising:
a tapered tip comprising a castellated sleeve, the castellated sleeve comprising a castellation portion comprising:
embrasures; and
merlons.

2. The stent-graft delivery system of claim 1 wherein said merlons are separated from one another by said embrasures.

3. The stent-graft delivery system of claim 1 wherein said embrasures are openings within said castellated sleeve between said merlons.

4. The stent-graft delivery system of claim 1 wherein a first embrasure of said embrasures separates a first merlon of said merlons from a second merlon of said merlons.

5. The stent-graft delivery system of claim 4 wherein said first embrasure is defined by a circumferential edge of said castellated sleeve, a first longitudinal edge of said castellated sleeve and a second longitudinal edge of said castellated sleeve.

6. The stent-graft delivery system of claim 5 wherein said circumferential edge extends along a circumference of said castellated sleeve between said first longitudinal edge and said second longitudinal edge.

7. The stent-graft delivery system of claim 6 wherein said first longitudinal edge extends between a proximal end of said castellation portion and said circumferential edge, and wherein said second longitudinal edge extends between said proximal end of said castellation portion and said circumferential edge.

8. The stent-graft delivery system of claim 4 wherein said first merlon is defined by a circumferential edge of said castellated sleeve, a first longitudinal edge of said castellated sleeve and a second longitudinal edge of said castellated sleeve.

9. The stent-graft delivery system of claim 8 wherein said circumferential edge extends along a circumference of said castellated sleeve between said first longitudinal edge and said second longitudinal edge, said circumferential edge being at a proximal end of said castellation portion.

10. The stent-graft delivery system of claim 9 wherein said first longitudinal edge extends between said circumferential edge and a distal end of said castellation portion, and wherein said second longitudinal edge extends between said circumferential edge and said distal end of said castellation portion.

11. The stent-graft delivery system of claim 1 wherein a radius of curvature of said merlons equals a radius of curvature of said castellated sleeve.

12. The stent-graft delivery system of claim 1 wherein said merlons have a minimum radius of curvature at proximal ends of said merlons.

13. The stent-graft delivery system of claim 12 wherein a radius of curvature of said merlons gradually increases distally from said proximal ends.

14. The stent-graft delivery system of claim 1 wherein said castellated sleeve further comprises a cylindrical wall portion, a distal end of said castellation portion being connected to a proximal end of said cylindrical wall portion.

15. The stent-graft delivery system of claim 14 wherein said tapered tip further comprises a primary sheet abutment surface connected to a distal end of said cylindrical wall portion.

16. A stent-graft delivery system comprising:
a tapered tap comprising a castellated sleeve, the castellated sleeve comprising a castellation portion comprising:
embrasures; and
merlons;
a spindle comprising:
a spindle body; and
spindle pins, wherein each spindle pin of said spindle pins extends from said spindle body to a respective merlon of said merlons.

17. The stent-graft delivery system of claim 16 further comprising a stent-graft comprising a proximal anchor stent ring comprising proximal apexes, wherein said proximal apexes are radially constrained by said merlons.

18. The stent-graft delivery system of claim 17 wherein each proximal apex of said proximal apexes extends around a respective spindle pin of said spindle pins.

19. The stent-graft delivery system of claim 18 wherein said proximal apexes are secured within a space between said spindle body and said merlons.

20. The stent-graft delivery system of claim 17 wherein said proximal anchor stent ring further comprises:
distal apexes; and
struts extending between said proximal apexes and said distal apexes, wherein a pair of said struts is aligned with a respective embrasure of said embrasures.

21. The stent-graft delivery system of claim 20 wherein said stent-graft further comprises a graft material attached to said distal apexes.

22. The stent-graft delivery system of claim 17 wherein said stent-graft further comprises a pair of anchor pins adjacent each proximal apex of said proximal anchor stent ring, wherein said anchor pins are radially constrained by said merlons.

23. The stent-graft delivery system of claim 22 wherein proximal ends of said merlons have a maximum curvature of said merlons to facilitate retention of said anchor pins.

24. A method of deploying a stent-graft comprising:
radially constraining proximal apexes of a proximal anchor stent ring of said stent-graft in a space between merlons of a castellated sleeve of a tip and a spindle, said spindle comprising spindle pins, said proximal apexes extending around said spindle pins;
radially constraining a graft material of said stent-graft in a primary sheath, a proximal end of said graft material being attached to distal apexes of said proximal anchor stent ring, said proximal anchor stent ring further comprising struts extending between said proximal apexes and said distal apexes;
retracting said primary sheath to allow said proximal end of said graft material and said distal apexes of said proximal anchor stent ring to radially expand, wherein said struts extend through embrasures of said castellated sleeve; and
advancing said tip to deploy said proximal apexes.

25. The method of claim 24 wherein anchor pins of said proximal anchor stent ring penetrate into a vessel wall upon deployment of said proximal apexes.

26. The method of claim 24 wherein said advancing comprises moving a castellated sleeve to expose said spindle pins.

27. The method of claim 26 wherein exposure of said spindle pins releases said proximal apexes of said proximal anchor stent ring.
